(19) United States
(12) Patent Application Publication
(10) Pub. No.: US 2018/0080070 A1
(43) Pub. Date: Mar. 22, 2018

(54) METHOD FOR TEMPLATE-DEPENDENT MULTIPLE DISPLACEMENT AMPLIFICATION

(71) Applicant: Saint Louis University, St. Louis, MO (US)

(72) Inventors: Xiaofeng FAN, St. Louis, MO (US); Adrian M. DI BISCEGLIE, St. Louis, MO (US)

(73) Assignee: Saint Louis University, St. Louis, MO (US)

(21) Appl. No.: 15/565,196

(22) PCT Filed: Apr. 1, 2016
(86) PCT No.: PCT/US16/25474

§ 371 Date: Oct. 9, 2017

Related U.S. Application Data
(60) Provisional application No. 62/144,664, filed on Apr. 8, 2015.

(51) Int. Cl. C12Q 1/68; C12Q 1/70

(52) U.S. Cl. CPC C12Q 1/6848; C12Q 1/6853; C12Q 1/707; C12Q 1/6876

(57) ABSTRACT

Provided herein are methods for template-dependent multiple displacement amplification (tdMDA) that preferably use 5′ blocked pentamer primers.

This application claims benefit of priority to U.S. Provisional Application 62/144,664, filed Apr. 8, 2015, the entire contents of which are hereby incorporated by reference.

The invention was made with government support under Grant No. R01 DK80711 awarded by the National Institutes of Health. The government has certain rights in the invention.

### BACKGROUND

### 1. Field

The present disclosure relates generally to the field of molecular biology. More particularly, it concerns methods of template-dependent multiple displacement amplification.

### 2. Description of Related Art

Multiple displacement amplification (MDA) is associated with two major concerns that diminish its power in nucleic acid amplification. Using random hexamer primer, MDA amplifies any DNA/cDNA in the template (Binga et al., 2008). Thus, potential contamination from reagents and experimental process is the first concern. This concern, however, could be minimized or eliminated by careful performance and reagent decontamination using ultraviolet (UV)-based approach (Woyke et al., 2011). Next, the generation of template-independent product, so called junk DNA, is another problematic issue associated with MDA. When the amount of template DNA/cDNA is low, efficient amplification of MDA requires higher concentration of random hexamer primers, which facilitates the production of junk DNA. As a consequence, junk DNA may account for up to 70% of MDA product even using extensively optimized protocols (Wang et al., 2013 and Pan et al., 2013). For the same reason, most commercial kits require a minimum of 10 ng of DNA/cDNA as starting templates in MDA. Junk DNA is also a factor contributing to amplification bias in MDA (Lasken 2007). This phenomenon is not specific to MDA but seems to be common in other phi29 DNA polymerase-based isothermal amplification methods, such as rolling circle amplification (RCA) (Dean et al., 2001).

Elimination of junk DNA has attracted significant investigation, including the use of additives [DMSO (Woyke et al., 2011 and Wang et al., 2004), T4 gene 32 (Wang et al., 2004), trehalose (Pan et al., 2008), single-stranded DNA binding protein (SSB) (Wu et al., 2006)], random hexamer primer containing C3 spacer (12), RNA primer (Takahashi et al., 2009), chimeric DNA-RNA primer (Kurn et al., 2005), modulation of incubation temperature (Alsmadi et al., 2009), and the reduction of reaction volume (Hutchison et al., 2005). However, none of these approaches has consistently achieved success.

### SUMMARY

Provided herein are methods for template-dependent multiple displacement amplification (tdMDA) that use 5′ blocked pentamer primers.

In one embodiment, there are provided compositions comprising a population of pentamer oligonucleotides with blocked 5′ ends. In some aspects, the oligonucleotides each comprise at least one phosphothioate linkage. In some aspects, the oligonucleotides each comprise at least two phosphothioate linkages. In various aspects, the at least one or two phosphothioate linkages may be the most 3′ backbone linkages in the pentamer. In some aspects, the oligonucleotides comprise a modification at the most 3′ backbone linkage that provides resistance to a 3′ to 5′ exonuclease activity. The blocked 5′ ends may be blocked by any means that prevents efficient replication slippage of a polymerase. In some aspects, the blocked 5′ ends comprise a dSpacer, a 5′ inverted dideoxynucleotide, a 5′ carbon chain spacer, or a 5′ ethyleneglycol spacer. In some aspects, each oligonucleotide in the population comprises at least one adenine, one guanidine, one cytidine, and one thymidine.

In some aspects, the population of oligonucleotides is homogeneous. In other aspects, the population of oligonucleotides is heterogeneous. In some aspects, a heterogeneous population of oligonucleotides comprises at least two distinct pentamer oligonucleotide sequences. In some aspects, the at least two distinct pentamer oligonucleotide sequences are present in equal proportion in the population. In some aspects, the at least two distinct pentamer oligonucleotide sequences are present in unequal proportion in the population.

In some aspects, the population of oligonucleotides comprises at least two, three, four, five, six, seven, eight, nine, 10, 11, 12, 13, 14, 15, 16, 17, 18, 19, 20, or more distinct pentamer oligonucleotides. In some aspects, the population of oligonucleotides has a concentration of 100 nM, 1 μM, 10 μM, 20 μM, 40 μM, 60 μM, 80 μM, 100 μM, 200 μM, 500 μM, 750 μM, 1 mM, 10 mM, 100 mM, 1 M or any value derivable therein.

In one embodiment, methods are provided for amplifying a target nucleic acid comprising: (a) obtaining the target nucleic acid; (b) adding at least one polymerase, 5′-blocked random pentamer oligonucleotides, and deoxynucleotide triphosphates (dNTPs) to the target nucleic acid; and (c) incubating the target nucleic acid under isothermal conditions to allow for amplification of the target nucleic acid. In some aspects, the at least one polymerase comprises a strand displacing nucleic acid polymerase. In some aspects, the strand displacing polymerase is a phi29 DNA polymerase. In some aspects, the at least one polymerase, 5′-blocked random pentamer oligonucleotides, and deoxynucleotide triphosphates (dNTPs) are known to be free of contamination.

In some aspects, the target nucleic acid is genomic DNA or cDNA. In some aspects, the target nucleic acid is pathogen DNA or cDNA. In some aspects, the target nucleic acid is a viral genome, such as, for example, an HCV, HIV, HPV, or influenza genome. In some aspects, the viral genome comprises about 0.001%, 0.01%, 0.05%, 0.1%, 0.5%, or 1% of the nucleic acid in the sample. In some aspects, the target nucleic acid is 1 kb, 2 kb, 5 kb, 10 kb, 20 kb, 50 kb, 100 kb, 1000 kb, 10,000 kb, 100,000 kb, 1,000,000 kb, 10,000,000 kb, or any range derivable therein.

In some aspects, the target nucleic acid is RNA. In these aspects, the method further comprises a step of reverse transcription (RT) of said RNA prior to step (b). In some aspects, the step of reverse transcription (RT) is performed using at least one 5′-blocked random pentamer oligonucleotide.

In some aspects, the target nucleic acid is extracted from a clinical specimen, such as, for example, a serum sample, a plasma sample, a tissue sample, or a hair sample. In some aspects, the clinical sample may be a cell-free sample. In some aspects, the target nucleic acid may be extracted from a single cell. In some aspects, the target nucleic acid is extracted from an environmental sample, such as, for example, a water sample, an air sample, or a soil sample.

In some aspects, the oligonucleotides each comprise at least one phosphothioate linkage. In some aspects, the oligonucleotides each comprise at least two phosphothioate linkages. In various aspects, the at least one or two phosphothioate linkages may be the most 3′ backbone linkages in the pentamer. The blocked 5′ ends may be blocked by any means that prevents efficient replication slippage of a polymerase. In some aspects, the blocked 5′ ends comprise a dSpacer, a 5′ inverted dideoxynucleotide, a 5′ carbon chain spacer, or a 5′ ethyleneglycol spacer. In some aspects, each oligonucleotide in the population comprises at least one adenine, one guanidine, one cytidine, and one thymidine.

In some aspects, the incubation of step (c) occurs at between about 25° C. and about 35° C., or any value derivable therein. In some aspects, the incubation of step (c) occurs at about 28° C. In some aspects, the incubation of step (c) occurs for between about 2 hours and about 23 hours. In some aspects, the incubation of step (c) occurs for between about 4 hours and about 22 hours, between about 8 hours and about 20 hours, between about 12 hours and about 18 hours, or between about 16 hours and about 18 hours, or any range or value derivable therein.

In some aspects, the methods may further comprise detecting the amplified target nucleic acids. In some aspects, detecting comprises sequencing the amplified target nucleic acids.

In one embodiment, there are provided amplified target nucleic acids produced according to a method of the present embodiments. In some aspects, the amplified target nucleic acid may be further defined as an amplified whole genome. In some aspects, the amplified target nucleic acid may be further defined as free of junk DNA. In some aspects, the amplified target nucleic acid may be further defined as an amplified pathogen genome.

In one embodiment, there are provided kits housed in suitable containers comprising a population of pentamer oligonucleotides according to the present embodiments, at least one polymerase, and dNTPs. In some aspects, the at least one polymerase is a phi29 DNA polymerase. In some aspects, the kit further comprises a reverse transcriptase.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. In particular there is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation of the method being employed to determine the value, or the variation that is tolerated by the method being employed. Also, the term “about” when used in conjunction with a stated numerical value or range denotes somewhat more or somewhat less than the state value or range, to within a range of ±10% of that stated. Unless otherwise clear from context, all numerical values provided herein are modified by the term about.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. Potential mechanisms for the production of junk DNA in MDA. High concentrations of random hexamer primers may facilitate the replication slippage to generate new DNA templates and therefore junk DNA. The blockage of primers' 5′ ends prevents the operation of replication slippage of phi29 DNA polymerase (A). Junk DNA may also be synthesized through an alternative mechanism (B). At a 30° C. incubation temperature in MDA, 4-bp binding may maintain a stable structure to initiate polymerization to generate 8-bp DNA fragment, which is able to serve as the template for continuous polymerization toward the accumulation of junk DNA.

### DESCRIPTION OF ILLUSTRATIVE EMBODIMENTS

Nucleic acid amplification is an indispensable technique in life science research. A well-known method for nucleic acid amplification is polymerase chain reaction (PCR). However, PCR cannot satisfy researchers' needs in many situations, particularly in amplification of large genomes or heterogeneous templates containing multiple DNA/cDNA species. In these settings, another amplification method, named multiple displacement amplification (MDA), has been developed (Dean et al., 2002). MDA is an isothermal amplification method based on phi29 DNA polymerase that has several exceptional features, including high processivity, strong strand displacement activity, and 3′-5′ exonuclease activity. These characteristics let MDA outperform PCR-based approaches in terms of amplification coverage, sensitivity, and fidelity (Nelson 2014). Owing to these advantages, MDA is now a standard amplification method in single-cell and global gene expression analysis. However, there is a long-standing issue associated with MDA, the generation of non-template product, or so called junk DNA, that significantly reduces the power of MDA. This issue has now been solved by a specially designed primer for use in MDA: 5′ blocked random pentamer primers.

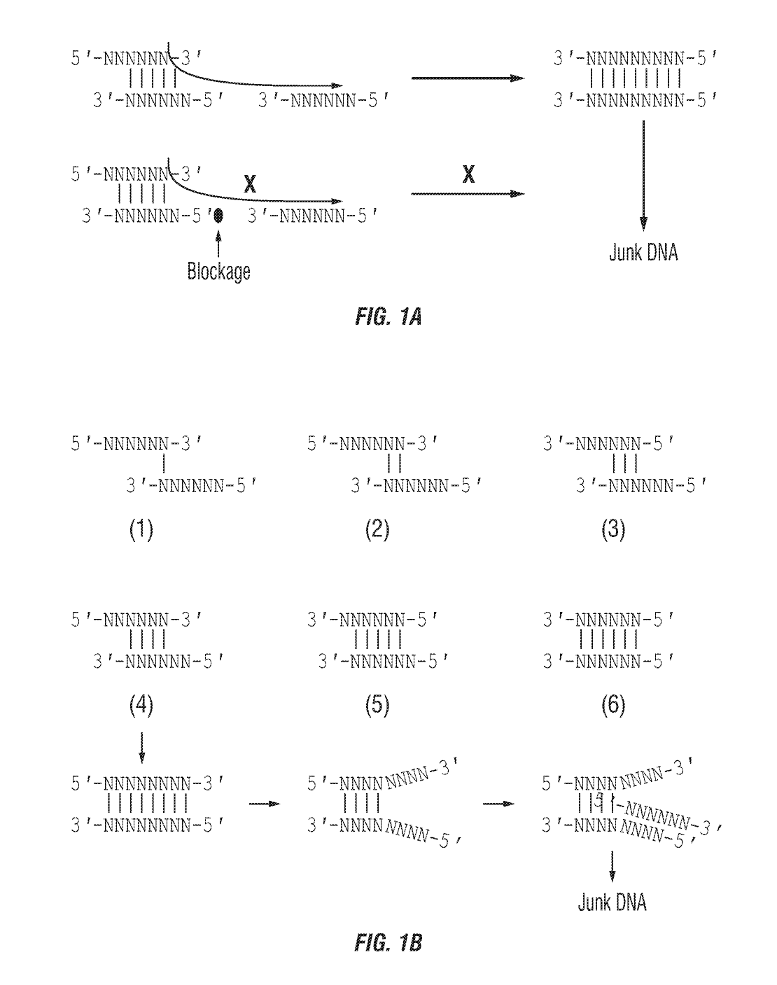

Phi29 DNA polymerase is associated with two salient features, strand-displacement activity and replication slippage (Hutchison et al., 2005). Without being bound by any theory, the later nature may allow a slippage of templates (hexamer primers) during polymerization, which produces new DNA strands to serve as new templates (FIG. 1A). To this point, it has been reported that phi29 DNA polymerase, unlike Taq DNA polymerases, is unable to serve as a biological brake (Sahu 2007). This process is thus stopped through the blockage at 5′ ends of primers, as described herein. Second, of six possible base-paired binding patterns among hexamers, the fourth pattern with 4-bp match between primers may start polymerization at both directions. Consequently, an 8-bp DNA fragment is synthesized. Upon partial melting, this fragment may form a “fork” structure to allow the binding of primers to initiate polymerization (FIG. 1B). Four base pairs of DNA binding appear to be stable and necessary for DNA synthesis in MDA. In contrast to hexamer primers, the maximum length of DNA produced among pentamer primers is 6 base pairs that are less possible to maintain a stable “fork” structure for subsequent polymerization. Without being bound by any theory, this might explain why only 5′ blocked pentamer primers, but not 5′ blocked hexamer primers, can suppress template-independent amplification in MDA.

### I. MDA

Multiple displacement amplification (MDA) is a non-PCR-based isothermal method based on the annealing of random sequence primers to denatured DNA, followed by strand-displacement synthesis at constant temperature (Blanco et al. J. Biol. Chem. 1989, 264, 8935-8940). It has been applied to samples with small quantities of genomic DNA, leading to the synthesis of high molecular weight DNA with limited sequence representation bias (Lizardi et al. Nature Genetics 1998, 19, 225-232; Dean et al., Proc. Natl. Acad. Sci. U.S.A. 2002, 99, 5261-5266). As DNA is synthesized by strand displacement, a gradually increasing number of priming events occur, forming a network of hyper-branched DNA structures. The reaction can be catalyzed by enzymes such as the phi29 DNA polymerase or the large fragment of the Bst DNA polymerase. The phi29 DNA polymerase possesses a proofreading activity resulting in error rates 100 times lower than Taq polymerase (Lasken et al., Trends Biotech. 2003, 21, 531-535).

In one particular form of the method, two sets of primers are used, a right set and a left set. Primers in the right set of primers each have a portion complementary to nucleotide sequences flanking one side of a target nucleotide sequence and primers in the left set of primers each have a portion complementary to nucleotide sequences flanking the other side of the target nucleotide sequence. The primers in the right set are complementary to one strand of the nucleic acid molecule containing the target nucleotide sequence and the primers in the left set are complementary to the opposite strand. The 5′ ends of primers in both sets are distal to the nucleic acid sequence of interest when the primers are hybridized to the flanking sequences in the nucleic acid molecule. Preferably, each member of each set has a portion complementary to a separate and non-overlapping nucleotide sequence flanking the target nucleotide sequence. Amplification proceeds by replication initiated at each primer and continuing through the target nucleic acid sequence. A key feature of this method is the displacement of intervening primers during replication. Once the nucleic acid strands elongated from the right set of primers reaches the region of the nucleic acid molecule to which the left set of primers hybridizes, and vice versa, another round of priming and replication will take place. This allows multiple copies of a nested set of the target nucleic acid sequence to be synthesized in a short period of time.

Multiple displacement amplification can be performed by (a) mixing a set of primers with a target sample, to produce a primer-target sample mixture, and incubating the primer-target sample mixture under conditions that promote hybridization between the primers and the target sequence in the primer-target sample mixture, and (b) mixing DNA polymerase with the primer-target sample mixture, to produce a polymerase-target sample mixture, and incubating the polymerase-target sample mixture under conditions that promote amplification of the target sequence. Strand displacement amplification is preferably accomplished by using a strand displacing DNA polymerase or a DNA polymerase in combination with a compatible strand displacement factor.

By using a sufficient number of primers in the right and left sets, only a few rounds of replication are required to produce hundreds of thousands of copies of the nucleic acid sequence of interest. For example, it can be estimated that, using right and left primer sets of 26 primers each, 200,000 copies of a 5,000 nucleotide amplification target can be produced in 10 minutes (representing just four rounds of priming and replication). It can also be estimated that, using right and left primer sets of 26 primers each, 200,000 copies of a 47,000 nucleotide amplification target can be produced in 60 minutes (again representing four rounds of priming and replication). These calculations are based on a polymerase extension rate of 50 nucleotides per second. It is emphasized that reactions are continuous and isothermal—no cycling is required.

The disclosed method has advantages over the polymerase chain reaction since it can be carried out under isothermal conditions. No thermal cycling is needed because the polymerase at the head of an elongating strand (or a compatible strand-displacement factor) will displace, and thereby make available for hybridization, the strand ahead of it. Other advantages of multiple strand displacement amplification include the ability to amplify very long nucleic acid segments (on the order of 50 kilobase pairs) as well as shorter segments (10 kilobase pairs or less). Long nucleic acid segments can be amplified in the disclosed method since there is no cycling that could interrupt continuous synthesis or allow the formation of artifacts due to re-hybridization of replicated strands. In multiple displacement amplification, single priming events at unintended sites will not lead to artificial amplification at these sites (since amplification at the intended site will quickly outstrip the single strand replication at the unintended site).

A. Target Sequence

The target sequence, which is the object of amplification, can be any nucleic acid. The target sequence can include multiple nucleic acid molecules, such as in the case of whole genome amplification, multiple sites in a nucleic acid molecule, or a single region of a nucleic acid molecule. For multiple displacement amplification, the target sequence may be a single region in a nucleic acid molecule or nucleic acid sample. For whole genome amplification, the target sequence is the entire genome or nucleic acid sample. For whole transcriptome amplification, the target sequence is cDNA generated from the entire population of mRNA present in a sample (e.g., a single cell). A target sequence can be in any nucleic acid sample of interest. The source, identity, and preparation of many such nucleic acid samples are known. It is contemplated that nucleic acid samples known or identified for use in amplification or detection methods be used for the method described herein. The nucleic acid sample can be a nucleic acid sample (e.g., DNA or RNA) isolated from serum. For multiple displacement amplification, particular target sequences are those that are difficult to amplify using PCR due to, for example, length or composition. For example, MDA may be used to amplify very limited amounts of pathogen DNA or cDNA in order to generate enough amount of product for subsequent pathogen-specific detection. In another example, MDA may be used to amplify DNA or RNA isolated from a single cell. In yet another example, MDA may be used to amplify DNA or RNA isolated from a cell-free sample (e.g. a cell-free serum sample).

Although multiple sites in a nucleic acid sample can be amplified simultaneously in the same MDA reaction, for simplicity, the following discussion will refer to the features of a single nucleic acid sequence of interest that is to be amplified. This sequence is referred to below as a target sequence. It is contemplated that a target sequence for MDA includes two types of target regions, an amplification target and a hybridization target. The hybridization target includes the sequence in the target sequence that is complementary to the primers in a set of primers. The amplification target is the portion of the target sequence that is to be amplified. For this purpose, the amplification target is preferably downstream of, or flanked by the hybridization target(s). There are no specific sequence or structural requirements for choosing a target sequence. The hybridization target and the amplification target within the target sequence are defined in terms of the relationship of the target sequence to the primers in a set of primers. The primers are designed to match the chosen target sequence. Although advantageous, it is not required that the sequence to be amplified and the sites of hybridization of the primers be separate since sequences in and around the sites where the primers hybridize will be amplified.

B. Primers

Primers for use in the disclosed amplification method are oligonucleotides having sequence complementary to the target sequence. Primers may also contain a blocked or modified 5′ end, such as, for example, an abasic site (e.g., dSpacer), an inverted dideoxy nucleotide (e.g., ddT), or a carbon spacer (e.g., C3 spacer or C18 spacer). In a set of primers, it is contemplated that the complementary portion of each primer may be complementary to a different portion of the target sequence. It is contemplated that the primers in the set be complementary to adjacent sites in the target sequence.

It is contemplated that, when hybridized to a target sequence, the primers in a set of primers are separated from each other. It is contemplated that, when hybridized, the primers in a set of primers are separated from each other by at least five bases. It is contemplated that, when hybridized, the primers in a set of primers are separated from each other by at least 10 bases, by at least 20 bases, by at least 30 bases, by at least 40 bases, or by at least 50 bases.

It is contemplated that, when hybridized, the primers in a set of primers are separated from each other by no more than about 500 bases, by no more than about 400 bases, by no more than about 300 bases, or by no more than about 200 bases. Any combinations of the upper and lower limits of separation described above are specifically contemplated, including all intermediate ranges. The primers in a set of primers need not, when hybridized, be separated from each other by the same number of bases.

The optimal separation distance between primers will not be the same for all DNA polymerases, because this parameter is dependent on the net polymerization rate. A processive DNA polymerase will have a characteristic polymerization rate that may range from 5 to 70,000 nucleotides per second, and may be influenced by the presence or absence of accessory ssDNA binding proteins and helicases. In the case of a non-processive polymerase, the net polymerization rate will depend on the enzyme concentration, because at higher concentrations there are more re-initiation events and thus the net polymerization rate will be increased. An example of a processive polymerase is phi29 DNA polymerase, which proceeds at 50 nucleotides per second. An example of a non-processive polymerase is Vent exo(−) DNA polymerase, which will give effective polymerization rates of four nucleotides per second at low concentration, or 16 nucleotides per second at higher concentrations.

To obtain an optimal yield in an MDA reaction, the primer spacing is preferably adjusted to suit the polymerase being used. Long primer spacing is preferred when using a polymerase with a rapid polymerization rate. Shorter primer spacing is preferred when using a polymerase with a slower polymerization rate. The following assay can be used to determine optimal spacing with any polymerase. The assay uses sets of primers, with each set made up of 5 left primers and 5 right primers. The sets of primers are designed to hybridize adjacent to the same target sequence with each of the different sets of primers having a different primer spacing. The spacing is varied systematically between the sets of primers in increments of 25 nucleotides within the range of 25 nucleotides to 400 nucleotides (the spacing of the primers within each set is the same). A series of reactions are performed in which the same target sequence is amplified using the different sets of primers. The spacing that produces the highest experimental yield of DNA is the optimal primer spacing for the specific DNA polymerase, or DNA polymerase plus accessory protein combination being used.

DNA replication initiated at the sites in the target sequence where the primers hybridize will extend to and displace strands being replicated from primers hybridized at adjacent sites. Displacement of an adjacent strand makes it available for hybridization to another primer and subsequent initiation of another round of replication. The region(s) of the target sequence to which the primers hybridize is referred to as the hybridization target of the target sequence.

A set of primers can include any desired number of primers of different nucleotide sequence. For MDA, it is contemplated that a set of primers include a plurality of primers. It is contemplated that a set of primers includes 3 or more, 4 or more, 5 or more, 6 or more, or 7 or more primers. In general, the more primers used, the greater the level of amplification that will be achieved. There is no fundamental upper limit to the number of primers that a set of primers can have. However, for a given target sequence, the number of primers in a set of primers will generally be limited to the number of hybridization sites available in the target sequence. For example, if the target sequence is a 10,000 nucleotide DNA molecule and 20 nucleotide primers are used, there are 500 non-overlapping 20 priming sites in the target sequence. Even more primers than this could be used if overlapping sites are either desired or acceptable. In general a set of primers includes no more than about 1024 primers, no more than about 500 primers, no more than about 300 primers, no more than about 200 primers, no more than about 100 primers, or no more than about 50 primers. A particular range is from 7 to about 50 primers. Any combination of the stated upper and lower limits for the number of primers in a set of primers described above is specifically contemplated, including all intermediate ranges.

A particular form of primer set for use in MDA includes two sets of primers, referred to as a right set of primers and a left set of primers. The right set of primers and left set of primers are designed to be complementary to opposite strands of a target sequence. It is contemplated that the complementary portions of the right set primers are each complementary to the right hybridization target, and that each is complementary to a different portion of the right hybridization target. It is contemplated that the complementary portions of the left set primers are each complementary to the left hybridization target, and that each is complementary to a different portion of the left hybridization target. The right and left hybridization targets flank opposite ends of the amplification target in a target sequence. It is contemplated that a right set of primers and a left set of primers each include a particular number of primers as described above for a set of primers. Specifically, generally, a right or left set of primers includes a plurality of primers. More often a right or left set of primers includes 3 or more primers. Still more often a right or left set of primers includes 4 or more, 5 or more, 6 or more, or 7 or more primers. It is contemplated that a right or left set of primers includes no more than about 200 primers, or no more than about 100 primers. In a particular embodiment, a right or left set of primers includes from 7 to about 100 primers. Any combination of the aforementioned upper and lower limits for the number of primers in a set of primers described above are specifically contemplated, including all intermediate ranges. It is also contemplated that, for a given target sequence, the right set of primers and the left set of primers include the same number of primers. It is also contemplated that, for a given target sequence, the right set of primers and the left set of primers are composed of primers of similar hybridization characteristics.

C. DNA Polymerase

DNA polymerases useful in the multiple displacement amplification must be capable of having strain-displacement nature, either alone or in combination with a compatible strand displacement factor, a hybridized strand encountered during replication. Such polymerases are referred to herein as strand displacement DNA polymerases. It is advantageous that a strand displacement DNA polymerase lack a 5′ to 3′ exonuclease activity. Strand displacement is necessary to result in synthesis of multiple copies of a target sequence. A 5′ to 3′ exonuclease activity, if present, might result in the destruction of a synthesized strand. It is also advantageous that DNA polymerases for use in the disclosed method are highly processive. The suitability of a DNA polymerase for use in the disclosed method can be readily determined by assessing its ability to cry out strand displacement replication. Exemplary strand displacement DNA polymerases are Bst large fragment DNA polymerase (Exo(−) Bst) and exo(−)Bca DNA polymerase, bacteriophage phi29 DNA polymerase, phage M2 DNA polymerase, phage phiPRD1 DNA polymerase, exo(−)VENT® DNA polymerase, Klenow fragment of DNA polymerase I, T5 DNA polymerase, Sequenase, PRD1 DNA polymerase, and T4 DNA polymerase holoenzyme. Bacteriophage phi29 DNA polymerase is a monomeric protein-primed DNA-dependent replicase belonging to the eukaryotic-type family of DNA polymerases (family B). A phi29 DNA polymerase also contains an exonuclease domain that catalyzes exonucleolysis of mismatched nucleotides.

Strand displacement can be facilitated through the use of a strand displacement factor, such as a helicase. It is considered that any DNA polymerase that can perform strand displacement replication in the presence of a strand displacement factor is suitable for use in the disclosed method, even if the DNA polymerase does not perform strand displacement replication in the absence of such a factor. Strand displacement factors useful in strand displacement replication include BMRF 1 polymerase accessory subunit, adenovirus DNA-binding protein, herpes simplex viral protein ICP8, single-stranded DNA binding proteins (SSB), phage T4 gene 32 protein, and calf thymus helicase.

The ability of a polymerase to carry out strand displacement replication can be determined by using the polymerase in a strand displacement replication assay. Such assays should be performed at a temperature suitable for optimal activity for the enzyme being used, for example, 30° C. for phi29 DNA polymerase, from 46° C. to 64° C. for exo(−) Bst DNA polymerase, or from about 60° C. to 70° C. for an enzyme from a hyperthermophilic organism. For assays from 60° C. to 70° C., primer length may be increased to 20 bases for random primers, or to 22 bases for specific primers. Another useful assay for selecting a polymerase is the primer-block assay. The assay consists of a primer extension assay using an M13 ssDNA template in the presence or absence of an oligonucleotide that is hybridized upstream of the extending primer to block its progress. Enzymes able to displace the blocking primer in this assay are useful for the disclosed method.

### II. Definitions

The term “amplification,” as used herein, refers to a process of multiplying an original quantity of a nucleic acid template of a certain sequence in order to obtain greater quantities of nucleic acid with the same sequence. However, amplification can be performed in a sequence-specific manner or in a sequence-independent manner.

The term “genome,” as used herein, generally refers to the complete set of genetic information in the form of one or more nucleic acid sequences, including text or in silico versions thereof. A genome may include either DNA or RNA, depending upon its organism of origin. Most organisms have DNA genomes while some viruses have RNA genomes. As used herein, the term “genome” need not comprise the complete set of genetic information.

The term “hexamer” as used herein refers to a polymer composed of six units. More specifically, the term hexamer is used to describe an oligonucleotide primer having six nucleotide residues. The term “pentamer” as used herein refers to a polymer composed of five units. More specifically, the term pentamer is used to describe an oligonucleotide primer having five nucleotide residues.

The term “hybridization,” as used herein refers to the process of joining two complementary strands of DNA or one each of DNA and RNA to form a double-stranded molecule through Watson and Crick base-pairing or pairing of a universal nucleobase with one of the four natural nucleobases of DNA (adenine, guanine, thymine, and cytosine).

The term “multiple displacement amplification” as used herein, refers to a non-PCR-based isothermal method based on the annealing of random hexamers to denatured DNA, followed by strand-displacement synthesis at constant temperature. It has been applied to small genomic DNA samples, leading to the synthesis of high molecular weight DNA with minimal sequence representation bias. As DNA is synthesized by involving strand displacement, a gradually increasing number of priming events occur, forming a network of hyper-branched DNA structures, usually dominant around 20 kb. The reaction can be catalyzed using enzymes such as the phi29 DNA polymerase or the large fragment of the Bst DNA polymerase.

The term “nucleic acid” as used herein, refers to a high-molecular-weight biochemical macromolecule composed of nucleotide chains that convey genetic information. The most common nucleic acids are deoxyribonucleic acid (DNA) and ribonucleic acid (RNA). The monomers that build nucleic acids are called nucleotides. Each nucleotide consists of three components: a nitrogenous heterocyclic base, either a purine or a pyrimidine (also known as a nucleobase); a pentose sugar; and a phosphate. Different nucleic acid types differ in the structure of the sugar in their nucleotides: DNA contains 2-deoxyribose while RNA contains ribose.

The term “polymerase” as used herein, refers to an enzyme that catalyzes the process of replication of nucleic acids. More specifically, DNA polymerase catalyzes the polymerization of deoxyribonucleotides alongside a DNA strand, which the DNA polymerase “reads” and uses as a template. The newly-polymerized molecule is complementary to the template strand and identical to the template's partner strand.

The term “primer,” as used herein refers to an oligonucleotide that is capable of acting as a point of initiation of synthesis when placed under conditions in which synthesis of a primer extension product, which is complementary to a nucleic acid strand, is induced (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products.

Preferably, the primer is an oligodeoxyribonucleotide. The primer must be sufficiently long to prime the synthesis of extension products in the presence of the inducing agent. The exact lengths of the primers will depend on many factors, including temperature, composition of primer, use of the method, and the parameters used for primer design, as disclosed herein.

The term “sensitivity” as used herein refers to a measure of the ability of a given reaction mixture to amplify very low quantities of DNA such as, quantities in the picogram range (1 picogram=1×10⁻¹² gram). For example, a given reaction mixture that produces a useful quantity of an amplified DNA in an amplification reaction starting from a given quantity of template DNA is more sensitive than another given reaction mixture that cannot produce a useful quantity of DNA from the same quantity of template DNA.

### III. Applications of the Present Embodiments

A. Whole Genome/Transcriptome Amplification

Whole Genome Amplification refers to an in vitro method that is used to amplify a genomic DNA sample and generate large amounts of amplified DNA for further molecular analyses. The described methods would work on any DNA of any origin, both from non-cellular sources (a virus, cell-free circulating RNA) and from cellular sources (single human cells, bacteria, archaea, eukaryotes). In cases where the target sequence is an RNA element, the RNA can be reverse transcribed into cDNA, and the resulting cDNA used in the methods provided herein. For WGA, random pentamer primers having a blocked 5′ end are used. Such primers may have the following structure: /5Sp18/NNN*N*N, wherein 5Sp18 is a 5′ C18 spacer and * represents a phosphothioate backbone linkage. In some cases, tdMDA may be used as a pre-amplification step prior to a specific PCR amplification.

B. Target-Enriched Amplification

One exemplary application of target-enriched amplification by tdMDA is ultra-sensitive pathogen detection within highly complex samples, such as, for example, viral, bacterial, or fungal genomes present in a clinical human sample or an environmental sample. Examples of pathogens that can be targeted by the present embodiments, include, without limitation, bacteria (e.g., Escherichia coli, Klebsiella pneumoniae, Klebsiella oxytoca, Serratia marcescens, Enterobacter cloacae, Enterobacter aerogenes, Proteus mirabilis, Pseudomonas aeruginosa, Acinetobacter baumannii, Stenotrophomonas maltophilia, Staphylococcus aureus, Staphylococcus epidermidis, Staphylococcus haemolyticus, Streptococcus pneumoniae, Streptococcus pyogenes, Streptococcus agalactiae, Streptococcus mitis, Enterococcus faecium, Enterococcus faecalis, Candida albicans, Candida tropicalis, Candida par apsilosis, Candida krusei, Candida glabrata, Mycobacterium tuberculosis, and Aspergillus fumigatus), fungi (e.g., Candida spp. including C. albicans, C. tropicalis, C. kern, C. krusei and C. galbrata; Aspergillus spp. including A. fumigatus and A. flavus; Cryptococcus neofornans; Blastomyces spp. including Blastomyces dermatitides; Pneumocystis carinii; Coccidioides immitis; Basidiobolus ranarum; Conidiobolus spp.; Histoplasma capsulatum; Rhizopus spp. including R. oryzae and R. microsporus; Cunninghamella spp.; Rhizomucor spp.; Paracoccidioides brasiliensis; Pseudallescheria boydii; Rhinosporidium seeberi; and Sporothrix schenckii), viruses (influenza virus, hepatitis C virus, human immunodeficiency virus, dengue virus, human papilloma virus, hepatitis B virus, ebola virus, yellow fever virus, etc.).

The present methods may also be used advantageously in a variety of other contexts, such as agriculture, environmental testing/ecology and forensics. Specimens related to any of these fields of examination can be assayed using whole genome/transcriptome amplification to generate information relevant to the area of investigation.

The embodiments described herein rely on the existence of one or more pentamer repeats within the target nucleic acid (e.g., pathogen genome). The repeated pentamer sequence(s) may be targeted by a specifically designed pentamer primer(s) thus resulting in enriched amplification of the target nucleic acid by tdMDA (in cases where the target nucleic acid is RNA, said nucleic acid may be reverse transcribed into cDNA prior to tdMDA using methods well known to those of skill in the art), which can then be used for detection of the target using further target-specific methods (e.g., real-time PCR, Sanger or next-generation sequencing). A pentamer repeat in a target nucleic acid may occur at least 5 times, at least 10 times, at least 15 times, at least 20 times, at least 25 times, at least 30 times, or at least 35 times within said genome. A pentamer repeat in a target nucleic acid is selected to provide relative genome specificity rather than absolute genome specificity in cases where the target is comprised within a complex nucleic acid sample. In these cases, other non-target nucleic acids in the sample are likely to also contain one or more occurrence of the selected pentamer repeat; however, if the target nucleic acid has a higher frequency of occurrence of the pentamer repeat than a non-target nucleic acid, then amplification of the target will be enriched relative to the non-target nucleic acid.

### IV. Kits

Certain aspects of the present disclosure may provide kits, such as kits for performing tdMDA. For example, a kit may comprise one or more 5′ blocked pentamer oligonucleotide compositions as described herein and optionally instructions for their use. Kits may also comprise one or more polymerases, such as phi29 DNA polymerase. In other embodiments, a subject kit may comprise pre-filled ampoules of 5′ blocked pentamer oligonucleotides, optionally lyophilized, for use in a tdMDA reaction.

Kits may comprise a container with a label. Suitable containers include, for example, bottles, vials, and test tubes. The containers may be formed from a variety of materials, such as glass or plastic. The container may hold a composition that includes 5′ blocked pentamer oligonucleotide that is effective for MDA applications, such as described above. The label on the container may indicate that the composition is used for a specific target, and may also indicate directions for use, such as those described above. The kit of the disclosure will typically comprise the container described above and one or more other containers comprising materials desirable from a commercial and user standpoint, including buffers, diluents, and package inserts with instructions for use.

### V. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Elimination of Junk DNA by Template-Dependent MDA

In a previous study, the composition of MDA product from the samples and negative controls (H₂O) were analyzed through pyrosequencing and bioinformatic analysis (Wang et al., 2013). There were no protein-coding signals detected in sequencing reads unmapped to the genome of any known organism. Therefore, random hexamer primers might be a major source for the formation of junk DNA. As phi29 DNA polymerase is able to bind and initiate polymerization of as few as 4 bp of oligonucleotide (Berman et al., 2007), random hexamer primers, at high concentration, may serve as the template for DNA synthesis. Such a process is accelerated by strand-displacement activity and replication slippage of phi29 DNA polymerase to form template-independent product, i.e., junk DNA (Nelson 2014).

As random hexamer primers are the most possible source for the formation of junk DNA, further efforts have been focused on the design of specific primers that only initiate template-dependent amplification. In doing so, the reagents used in MDA were first ensured to be free of potential contamination. Then these “clean” reagents were used to test the efficiency of specially designed primers in terms of the elimination of junk DNA in MDA. In both phases, a standard MDA experiment was set up as follows: 0.0001-30 ng of template nucleic acid; 1-100 μM random hexamer (5′-NNNN*N*N-3′; N=A/G/C/T; *=phosphothioate bond); 0.25 mM dNTPs; phi29 DNA polymerase; and buffer. Because phi29 DNA polymerase has exonuclease activity, random hexamer primers contain phosphothioate bonds for the last two backbone linkages to prevent digestion during MDA. A hepatitis C virus genome (˜9 kb) cloned from the long RT-PCR amplicon, named LRP (Fan and Di Bisceglie, 2010), was used as the positive control. Sterile water, purchased from Sigma and further treated by 10-minute UV irradiation, was used as the negative control.

Testing reagents for contamination: phi29 DNA polymerases from four suppliers, including New England Biolabs (Catalog M0269L), Epicentre (Catalog P040110), Lucigen (Catalog 30221), and Thermo Scientific (Catalog EP0091) were tested. After a 16-hour incubation at 30° C. with a low concentration of random hexamer primers (2 μM), DNA bands on 1% agarose gel were much weaker in MDA using phi29 DNA polymerase from Epicentre than the phi29 DNA polymerases from other three suppliers. Therefore, phi29 DNA polymerase from Epicentre was chosen for further studies.

Testing primer designs in template-dependent MDA: A total of 31 primer designs were divided into seven groups (Table 1). Each primer was estimated at various concentrations from 2 μM to 100 μM in MDA with either positive or negative controls. Primers containing locked nucleic acid were purchased from Exiqon (Woburn, USA). All other primers were synthesized by Integrated DNA Technologies (Coralville, USA).

Group 1 Primers:

Phosphothioate bonds were placed in different positions to determine the effect on the ability of a primer to initiate DNA polymerization. It was found that phosphothioate bonds are required to resist exonuclease activity of phi29 DNA polymerase.

Group 2 Primers:

This group of primers was modified with at least one C3 spacer that prevents efficient self-binding among random hexamer primers. Primers FanC3, FanC4, and 6NS2C3_5 completely abolished MDA. Primers FanC1 and FanC2 reduced polymerization in comparison to the primers without C3 spacer, but still produced product in a template-independent manner.

Group 3 Primers:

This group of primers was modified with a C3 spacer and a locked nucleic acid (LNA) to stabilize the binding between templates and primers. Only primer FanC8 initiated polymerization, but in both positive and negative controls.

Group 4 Primers:

This group of primers was RNA primers with or without C3 spacer. Junk DNA was generated, particularly in the high primer concentration.

Group 5 Primers:

This group of primers was constrained and had two phosphothioate bonds. Reduction of primer randomness resulted in decrease of MDA product. However, none of them eliminated the generation of junk DNA.

Group 6 Primers:

This group of primers was random hexamer blocked at their 5′ end. At low concentrations, hexamer primers efficiently suppressed the synthesis of junk DNA without any impact on template-dependent amplification. However, at high concentrations, template-independent amplification in the negative controls was observed. Advanced purification of the primer by HPLC had no effect on MDA.

TABLE 1

| | | | | MDA | | | | |
|---|---|---|---|---|---|---|---|---|
| | | | | | Template | | | |
| | | | | | LRP (1 ng) | | H₂O | |
| | | | | | Primer concentration | | | |
| No | Group | Primer | Sequence (5′→3′) | Incubation | 10 μM | 50 μM | 10 μM | 50 μM |
| 1 | 1 | 6N | NNNNNN | 30 C | − | − | − | − |
| 2 | | 6NS2 | NNNN*N*N | 30 C | ++++ | ++++ | +++ | ++++ |
| 3 | | FanC5 | NN*N* NNN | 30 C | − | − | − | − |
| 4 | | FanC6 | N*N*N NNN | 30 C | − | − | − | − |
| 5 | | FanC7 | NNN*N*NN | 30 C | +++ | ++++ | +++ | ++++ |
| 6 | 2 | FanC1 | N/iSpC3/NNN*N*N | 30 C | ++ | +++ | ++ | +++ |
| 7 | | FanC2 | NN/iSpC3/NN*N*N | 30 C | − | ++ | − | ++ |
| 8 | | FanC3 | NNN/iSpC3/N*N*N | 30 C | − | − | − | − |
| 9 | | FanC4 | NNNN/iSpC3/*N*N | 30 C | − | − | − | − |
| 10 | | 6NS2C3_5 | N/iSpC3N/iSpC3//iSpC3/ | 30 C | − | − | − | − |
| | | | N/iSpC3/N*/iSpC3/*N | | | | | |
| 11 | 3 | FanC8 | NNN/iSpC3/N*+N*NN | 30 C | − | − | − | − |
| 12 | | FanC9 | NNN/iSpC3/+N*NNN | 30 C | − | − | − | − |
| 13 | | FanC10 | NNNN/iSpC3/+N*NN | 30 C | − | − | − | − |
| 14 | 4 | 6RNS5 | rN*rN*rN*rN*rN*rN | 30 C | +++ | ++++ | + | +++ |
| 15 | | 6RNS5C3_5 | rN*/iSpC3/*rN1*/iSpC3/ | 30 C | − | − | − | − |
| | | | *rN1*/iSpC3/*rN*/iSpC3/ | | | | | |
| | | | *rN*/iSpC3/*rN | | | | | |
| 16 | 5 | FanC11 | RRR*R*RR | 30 C | ++ | +++ | ++ | +++ |
| 17 | | FanC20 | YYY*Y*YY | 30 C | + | +++ | − | ++ |
| 18 | | FanC12 | NWN*N*SN | 30 C | + | ++ | + | ++ |
| 19 | | FanC13 | NSN*N*WN | 30 C | + | ++ | + | ++ |
| 20 | | FanC14 | WNN*N*SN | 30 C | + | ++ | + | ++ |
| 21 | | FanC15 | SNN*N*WN | 30 C | + | ++ | + | ++ |
| 22 | 6 | FanC18 | /5InvddT/NNN*N*NN | 30 C | +++ | ++++ | − | ++ |
| 23 | | FanC19 | /5InvddT/NNN*N*NN | 30 C | +++ | ++++ | − | ++ |
| | | | (HPLC purification) | | | | | |
| 24 | | FanC21 | TNNNN*N*N | 30 C | +++ | ++++ | +++ | ++++ |
| 25 | 7 | FanC22 | /5dSp/idSp/NNN*N*N | 28 C | − | − | − | − |
| 26 | | FanC23 | /5InvddT/NNN*N*N | 28 C | ++++ | ++++ | − | + |
| 27 | | FanC24 | /5dSp/NNN*N*N | 28 C | ++++ | ++++ | − | − |
| 28 | | FanC25 | NNN*N*N | 28 C | ++++ | ++++ | +++ | ++++ |
| 29 | | FanC26 | /5SpC3/NNN*N*N | 28 C | ++++ | ++++ | − | − |
| 30 | | FanC27 | /5SpC18/NNN*N*N | 28 C | ++++ | ++++ | − | − |
| 31 | | FanC28 | /5SpC18/NN*N*NN | 28 C | + | + | − | − |

Each primer was estimated at multiple concentrations ranging from 2 μM to 100 μM. MDA results are presented with primer concentrations at 10 μM and 50 μM, presumly defined as low concentration and high concentration, respectively. Degenerate bases are matched with standard International Union of Pure and Applied Chemistry (IUPAC) codes. MDA was performed in 50 μL of reaction and incubated at 28° C. (the group 7 primers) or 30° C. (all other primers) for 16 hours. An aliquot of 10 μL MDA product was run on 1% agarose gel. Yield of MDA product was judged based on the strength of DNA bands on the gel from invisible (−) to 4 plus (++++). The 4 plus (++++) corresponds to five micrograms of MDA product as determined with NanoDrop spectrophotometer.

Group 7 Primers:

This group of primers was random pentamer primers blocked at their 5′ end. Primer FanC22, doubly blocked at its 5′ end, abolished MDA. Primers blocked with inverted, 2′,3′ dideoxy-dT base (5′ Inverted ddT) (Primer FanC23), dSpacer (FanC24), and C3 spacer (FanC26) all eliminated junk DNA at low concentrations. At high primer concentrations, primers FanC24 and FanC26 eradicated the template-independent amplification. These two primers, either dissolved in water or TE (Tris-Ethylenediaminetetraacetic acid) buffer, were further estimated for the reproducibility that involved a multi-round thaw-freeze procedure. Among 20 experimental repeats, four (20%) experiments showed weak DNA bands on 1% agarose gel. There was no difference in terms of the solvent used to dissolve primers (water or TE buffer). However, this was not observed in random pentamer primers with their 5′ end blocked using C18 spacer (FanC27) up to 40 experimental repeats. Hence, the lack of high reproducibility in both Fan24 and Fan26 cannot be attributed to the instability of both dSpacer and C3 spacer as suggested in a previous study (Brukner et al., 2005). Instead, dSpacer and C3 spacer, but not C18 spacer, cannot completely block occurrence of replication slippage perhaps due to limited steric hindrance. The use of primer FanC28, one position move of phosphothioate bonds toward the 5′end, resulted in 90% reduction of MDA yield.

There are multiple ways to block 5′ ends of primers. The use of inverted ddT is efficient but introduces an additional base that promotes the generation of junk DNA at high concentration as seen with primer FanC23. Primers FanC24 and FanC26, blocked respectively with a dSpacer and a C3 spacer, remain the lack of high reproducibility. Primer FanC27, blocked with a C18 spacer, yielded the best and consistent results.

In the negative controls, no visual DNA was observed with the use of the primer FanC27 up to 200 μM. However, amplification efficiency was reduced at primer concentrations higher than 140 μM. Very high concentrations of the primer may consume magnesium ion and therefore decrease the activity of phi29 DNA polymerase. In practice, primer concentrations are dependent on the amount of template. In these experiments, 80 μM of FanC27 was enough to generate 2 μg of DNA in a typical 50-4, MDA with 1 pg of LRP (positive control) as the template. The incubation temperature should be adjusted onto 28° C. to maximize amplification efficiency in MDA using the pentamer primer FanC27.

### Example 2—Enriched Amplification of the HCV Genome

Enriched amplification of HCV RNA (eaHCV) is one of applications of tdMDA. Briefly, the primers designed for tdMDA consist of five random nucleic bases (5′-NNN*N*N-3′) with a 5′ C18 spacer, where the * represents phosphothioate backbone linkages. As described above, the elimination of template-independent amplification (or junk DNA) depends on the special modifications rather than nucleic bases. Using in-house perl scripts, HCV-specific primers have been designed through analyzing 5-bp repeats in 161 full-length HCV genomes retrieved from the Los Alamos HCV database. In a single HCV genome, regardless of HCV genotype, two pentamers (5′-ACTGG-3′ and 5′-AGCTG-3′) containing all four nucleic bases, A, G, C and T, were found to be repeated on average 31.1 and 37.9 times, respectively. In spite of the lack of strict HCV specificity, tdMDA with the use of these two pentamers, called HCV pentamer mix, which served as both RT and tdMDA primers, gave an enriched amplification of HCV genomes from complex targets, i.e., total RNA extracted from patient serum using, for example a miRNeasy Serum/Plasma kit (Qiagen). After reverse transcription (RT), the resulting cDNA was directly used as a template in tdMDA. The product of tdMDA (i.e., the product of eaHCV), can be subjected to sequencing (e.g., sequencing on an Illumina MiSeq or pyrosequencing) or HCV-specific PCR targeting to a definite genome domain, such as, for example, 5′ UTR, hypervariable region 1 (HVR1), NS3, NS5a, or NS5b.

The sensitivity of eaHCV-mediated HCV detection has been estimated in three different situations. First, using ten serum samples with known HCV RNA titers, 10-fold serial dilutions of total RNA extracted with Qiagen kits were generated, followed by HCV detection using either the eaHCV-based approach or conventional “nested” RT-PCR protocol containing a total of 70 PCR cycles. All ten samples detected HCV in two dilutions after negative RT-PCR results. Six samples were even positive after an additional three dilutions. Second, HCV RNA was detectable at the end of the treatment in ten patients relapsed from peg-IFN/ribavirin therapy (Chambers et al., 2005). However, no HCV was detected at the same time point in ten patients achieving sustained virological response. Finally, in a patient relapsed from combination therapy of telaprevir, peg-IFN and ribavirin, HCV RNA was detected by the eaHCV-based approach at the end of treatment. Taken together, these data demonstrate the utilization of the eaHCV-mediated approach in the detection of ultra-low HCV RNA load.

### Example 3—Virome Analysis in ALF Patients with Unknown Etiology

Nearly 12% patients with acute liver failure (ALF) have no etiologies identified in spite of exhaustive examinations. Using tdMDA, we conducted a virome analysis in 10 ALF cases from NIH-sponsored acute liver failure study group (ALFSG). Both RNA and DNA were extracted from 140 μl of patient serum, followed by reverse transcription, tdMDA and Illumina sequencing. Sequencing data was analyzed using the bioinformatics pipelines as we described previously. Categorization with NIH viral reference database showed the detection of known viruses in these patients, including Torque tenovirus (single-strand DNA virus), human endogenous retrovirus K113 (human provirus) and bacteriophage (double-strand DNA virus). Again, these experiments demonstrated the feasibility of tdMDA to be applied to clinical specimen containing ultralow amounts of genetic material.

TABLE 2
The Detection of All Known Viruses in 10 ALF Patients with Unknown
Etiology

| | | Read support | |
|---|---|---|---|
| Patients | Virus | Number | % |
| 1 | Enterobacteria phage P1 | 91,470 | 99.99 |
| | Human endogenous retrovirus K113 | 8 | 0.01 |
| 2 | Human endogenous retrovirus K113 | 1,632 | 100 |
| 3 | Human endogenous retrovirus K113 | 512 | 45.76 |
| | Enterobacteria phage M13 | 410 | 36.64 |
| | Bacteriophage f1 | 197 | 17.61 |
| 4 | Human endogenous retrovirus K113 | 1,165 | 28.85 |
| | Torque teno virus | 2,873 | 71.15 |
| 5 | Human endogenous retrovirus K113 | 734 | 100 |
| 6 | Human endogenous retrovirus K113 | 547 | 100 |
| 7 | Human endogenous retrovirus K113 | 646 | 97.14 |
| | Torque teno virus | 19 | 2.86 |
| 8 | Human endogenous retrovirus K113 | 783 | 90.31 |
| | Torque teno virus | 84 | 9.67 |
| 9 | Human endogenous retrovirus K113 | 810 | 69.65 |
| | Torque teno virus | 353 | 30.35 |
| 10 | Human endogenous retrovirus K113 | 678 | 100 |

Read support showed the number of sequencing reads mapped onto each virus as well as corresponding percentages among total reads mapped onto NIH viral reference database.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. References

The following references, to the extent that they provide exemplary procedural or other details supplementary to those set forth herein, are specifically incorporated herein by reference.
Alsmadi et al., BMC Res Notes., 2:48, 2009.
Berman et al., EMBO J., 26:3494-505, 2007.
Binga et al., ISME J., 2:233-41, 2008.
Brukner, Anal Biochem., 339:345-7, 2005.
Chambers et al., J. Virol., 79:3071-3083, 2005.
Dean et al., Genome Res., 11:1095-9, 2001.
Dean et al., Proc Natl Acad Sci USA, 99:5261-6, 2002.
Fan and Di Bisceglie, In RT-PCR Protocols (2nd edition) in the series Methods in Molecular Biology. Humana Press., 630:139-149, 2010.
Hutchison et al., Proc Natl Acad Sci USA., 102:17332-6, 2005.
Kurn et al., Clin. Chem., 51:1973-81, 2005.
Lasken, Curr Opin Microbiol., 10:510-6, 2007.
Nelson, Curr Protoc Mol Biol., 105:Unit 15.13, 2014.
Pan et al., Proc Natl Acad Sci USA., 105:15499-504, 2008.
Pan et al., Proc Natl Acad Sci USA., 110:594-9, 2013.
Sahu, DNA-based self-assembly and nanorobotics: theory and experiments. PhD thesis. Duke University, 2007.
Takahashi et al., Biotechniques, 47:609-15, 2009.
Wang et al., Biochem Biophys Res Commun, 436:525-529, 2013.
Wang et al., Genome Res., 14:2357-66, 2004.
Woyke et al., PLoS One., 6:e26161, 2011.
Wu et al., Appl Environ Microbiol., 72:4931-41, 2006.

1. A composition comprising a population of pentamer oligonucleotides with blocked 5′ ends.

2. The composition of claim 1, wherein the oligonucleotides each comprise at least one phosphothioate linkage.

3. The composition of claim 2, wherein the oligonucleotides each comprises at least two phosphothioate linkages.

4. The composition of claim 1, wherein the blocked 5′ ends comprise a dSpacer, a 5′ inverted dideoxynucleotide, a 5′ carbon chain spacer, a C3 spacer, a C18 spacer, or a 5′ ethyleneglycol spacer.

5. The composition of claim 1, wherein the population is homogeneous.

6. The composition of claim 1, wherein the population is heterogeneous.

7-9. (canceled)

10. The composition of claim 1, wherein the oligonucleotides each comprise at least one adenine, one guanidine, one cytidine, and one thymidine.

11. A method of amplifying a target nucleic acid comprising:
(a) obtaining the target nucleic acid;
(b) adding at least one polymerase, 5′-blocked random pentamer oligonucleotides, and deoxynucleotide triphosphates (dNTPs) to the target nucleic acid; and
(c) incubating the target nucleic acid under isothermal conditions to allow for amplification of the target nucleic acid.

12. The method of claim 1, wherein the at least one polymerase comprises a strand displacing nucleic acid polymerase.

13. (canceled)

14. The method of claim 11, wherein the target nucleic acid is genomic DNA or cDNA.

15. (canceled)

16. The method of claim 11, wherein the target nucleic acid is a viral genome.

17. The method of claim 11, wherein the viral genome comprises about 0.01% of the nucleic acid in the sample.

18. The method of claim 11, wherein the target nucleic acid is RNA.

19-20. (canceled)

21. The method of claim 11, wherein the target nucleic acid is extracted from a clinical sample.

22-23. (canceled)

24. The method of claim 11, wherein the target nucleic acid is extracted from a single cell.

25. The method of claim 11, wherein the 5′-blocked random pentamer oligonucleotides each comprise at least one phosphothioate linkage.

26. (canceled)

27. The method of claim 11, wherein the 5′-blocked random pentamer oligonucleotides each comprise a 5′ dSpacer, a 5′ inverted dideoxynucleotide, a 5′ carbon chain spacer, or a 5′ ethyleneglycol spacer.

28. The method of claim 11, wherein the incubation of step (c) occurs between about 25° C. and about 35° C.

29. (canceled)

30. The method of claim 11, wherein the incubation of step (c) occurs for between about 2 hours and about 23 hours.

31. (canceled)

32. The method of claim 11, further comprising detecting the amplified target nucleic acids.

33. (canceled)

34. An amplified target nucleic acid produced according to the method of claim 11.

35-36. (canceled)

37. A kit comprising a population of pentamer oligonucleotides according to any one of claims 1-9, at least one polymerase, and dNTPs.

38-39. (canceled)
